(19) United States
(12) Patent Application Publication
(10) Pub. No.: US 2026/0102252 A1
(43) Pub. Date: Apr. 16, 2026

(54) Conical Patella Resurfacing

(71) Applicant: Howmedica Osteonics Corp., Mahwah, NJ (US)

(72) Inventors: Geoff Westrich, New York, NY (US); Gokce Yildirim, Weehawken, NJ (US)

(21) Appl. No.: 19/419,917

(22) Filed: Dec. 15, 2025

Related U.S. Application Data
(63) Continuation of application No. 16/165,342, filed on Oct. 19, 2018, now Pat. No. 12,533,239.
(60) Provisional application No. 62/581,081, filed on Nov. 3, 2017.

(51) Int. Cl. A61F 2/38; A61B 17/16; A61B 17/17; A61B 34/10; A61F 2/30

(52) U.S. Cl. CPC A61F 2/3877; A61B 17/1677; A61B 17/1703; A61B 17/1767; A61F 2/30749; A61F 2/30771; A61B 17/1659; A61B 2034/102; A61F 2002/30125; A61F 2002/30205; A61F 2002/3092; A61F 2002/3881

(57) ABSTRACT

Disclosed herein are patellar implants and methods to prepare bone for receiving the same. The patellar implant may include an articulating surface and an anterior surface with a non-planar surface to engage with a resected patella. The non-planar surface may include a conically shaped portion to receive the resected patellar bone. A method for resurfacing a patella to implant a patellar implant having a recessed anterior surface may include the steps of determining the minimum implant and resection depth, medial and lateral patellar slope angles to select and resect with a symmetrical reamer.

### CROSS-REFERENCE TO RELATED APPLICATIONS

This application is a continuation of U.S. application Ser. No. 16/165,342, filed on Oct. 19, 2018, which claims the benefit of the filing of U.S. Provisional Patent Application No. 62/581,081, filed Nov. 3, 2017, the disclosures of which are hereby incorporated herein by reference in their entirety.

### FIELD OF INVENTION

The present invention relates to patellar implants and methods for bone preparation, and in particular to a patellar implant with a recess and methods for preparing bone to receive the same.

### BACKGROUND OF THE INVENTION

Patella resurfacing is routinely performed for treatments of various knee ailments including arthritis. After resurfacing the patella, a surgeon may implant a patellar implant on the resurfaced patella. The patellar implant generally has a patella contacting surface for engaging the resurfaced patella and an opposite surface that articulates with a distal end of a femoral body. The patella contacting surface is generally planar in shape to allow for attachment with a resected patella. A properly sized and configured patellar implant is required to ensure proper patellofemoral kinematics and prevent detachment from the natural patellar bone.

Bone interfacing geometries between the patellar implant and the resected patella generally consist of simple planar shapes because of the complexities of resecting patellar bone. Simple planar shapes limit patella implant design and may prevent patellar implant designs with varying thickness. For example, increasing the thickness of the patellar implant in regions of high mechanical stress and decreasing the thickness of the patellar implant in regions where maximizing bone preservation is desired are generally not possible on account of the simple planar shapes at the bone interfacing surfaces. Furthermore, patellar resurfacing is generally performed with little assistance from anatomical landmark registration. It is common to use only articulating surface high points as anatomical landmarks in conjunction with cutting clamps to perform the resection. This may result in improper patella sizing and increase the risk of patellar implant detachment.

Patellar implants are typically secured to a resected patella surface by either an onlay or an inlay technique. An onlay technique utilizes fixation features such as posts extending from the patellar implant which are received in corresponding recesses on the resected patella surface. Onlay technique, however, requires extensive patellar bone removal and may consequently weaken the resected patella and increase the risk of patella fracture. An inlay technique is performed by resecting a footprint of the patellar implant on the resecting surface and press-fitting the patellar implant into this recess. While the inlay technique may require less patellar bone removal, the patellar implant may not be embedded deep enough into the patella to provide the same level of fixation as achieved by utilizing an onlay technique. Further, the inlay technique may result in sharp, jagged bone fragments along the interface with the patellar implant and may consequently damage the patellar implant.

Therefore, there exists a need for improved patellar implants and methods for attaching the same to prepared bone.

### BRIEF SUMMARY OF THE INVENTION

Disclosed herein are patellar implants and methods for bone preparation to secure the same.

In a first aspect of the present invention, a patellar implant is provided. The patellar implant may include a posterior articulating surface an anterior surface for engaging a resected patellar bone. The posterior articulating surface may engage a femoral body. The anterior surface may be opposed to the posterior surface. The anterior surface may include a conically shaped portion. The conically shaped portion may extend and taper towards the posterior surface such that the resected patellar bone may be received in the conically shaped portion to secure the patellar implant to the resected patellar bone.

In accordance with the first aspect, the anterior surface may include a flat peripheral base disposed around a circumference of the conically shaped portion. A thickness of the resected patellar bone may be disposed within the conically shaped portion when the patellar implant is secured to the resected patellar bone. The thickness may be defined by a distance between a base and an apex of the conically shaped portion. The conically shaped portion may include at least one projection extending away from the anterior surface. The anterior surface may be a porous layer. A solid layer may be disposed between the porous layer and the polymeric layer.

Further in accordance with the first aspect, the posterior articulating surface may be symmetrically shaped to the anterior surface. The posterior articulating surface may include a median ridge portion extending posteriorly to and disposed between a lateral portion and a medial portion. The median ridge portion may extend along a first length in a superior to inferior direction and along a second length in a medial to lateral direction of the articulating surface. The first length may be greater than the second length. The median ridge portion may be elliptical in shape. The first length may be a major axis and the second length may be a minor axis of the median ridge portion respectively. The major axis may define a boundary between a medial side and a lateral side of the median ridge. The medial side may have one or more curves defined by a curve center located laterally to the major axis. The lateral side may have one or more curves defined by a curve center located medially to the major axis.

In a second aspect of the present invention, a patellar implant is provided. The patellar implant may include a posterior articulating surface for engaging a femoral body and an anterior surface for engaging a resected patellar bone. The anterior surface may include a recessed portion. The recessed portion may extend and taper towards the posterior surface such that the resected patellar bone may be received in the recessed portion to secure the patellar implant to the resected bone. The posterior articulating surface may include a median ridge portion extending posteriorly to and disposed between a lateral portion and a medial portion. The median ridge portion may extend along a first length in a superior to inferior direction and along a second length in a medial to lateral direction of the articulating surface. The first length may be greater than the second length.

In accordance with the second aspect, the median ridge portion may be elliptical in shape. The first length may be a major axis and the second length may be a minor axis of the median ridge portion respectively. The major axis may define a boundary between a medial side and a lateral side of the median ridge. The medial side may have one or more curves defined by a curve center located laterally to the major axis. The lateral side may have a one or more curves defined by a curve center located medially to the major axis.

A third aspect of the present invention is a method resurfacing a patellar bone to receive a patellar implant having a recessed anterior. A method in accordance with this aspect of the invention may include the steps of obtaining imaging of a patellar bone, determining from the obtained imaging a minimum implant depth of the patellar implant, determining optimum medial and lateral patellar slope angles of the patellar implant, selecting a symmetrical reamer and resecting the patellar bone. The minimum implant depth may be defined by a distance between a base and an apex of the recessed anterior surface of the patellar implant. Selecting a symmetrical reamer may be based on the minimum implant depth and medial-lateral patellar slope angles. The patellar bone may be resected with the symmetrical reamer such that the recessed anterior surface of the patellar implant may encompass at least a portion of the resected patellar bone. The step of determining a minimum implant depth of the conical patellar implant may include determining a patient specific patellar tilt angle.

### BRIEF DESCRIPTION OF THE DRAWINGS

A more complete appreciation of the subject matter of the present invention and the various advantages thereof may be realized by reference to the following detailed description, in which reference is made to the following accompanying drawings:

FIG. 1 is a bottom perspective view of a patellar implant according to a first embodiment of the present invention;

FIG. 2 is a front cross-sectional view along line A-A of the patellar implant of FIG. 1;

FIG. 3 is a side view of the patellar implant of FIG. 1 in conjunction with a resected patella;

FIG. 4 is a top view of the patellar implant of FIG. 1;

FIG. 5 is a side view of a patellar implant and a resected patella according to another embodiment of the present invention;

FIGS. 6A and 6B are schematic side views of a patella showing steps for patellar resection according to an embodiment of the present invention;

FIG. 6C shows a schematic side view of a patella showing steps for patellar measurement according to another embodiment of the present invention;

FIG. 7 is side cross-sectional view along line B—B of the patellar implant of FIG. 4; and

FIG. 8 is a side view of a symmetrical reamer for patella preparation.

### DETAILED DESCRIPTION

In describing preferred embodiments of the disclosure, reference will be made to directional nomenclature used in describing the human body. It is noted that this nomenclature is used only for convenience and that it is not intended to be limiting with respect to the scope of the invention.

As used herein, when referring to bones or other parts of the body, the term “anterior” means toward the front part or the face and the term “posterior” means toward the back of the body. The term “medial” means toward the midline of the body and the term “lateral” means away from the midline of the body. The term “superior” means closer to the head and the term “inferior” means more distant from the head.

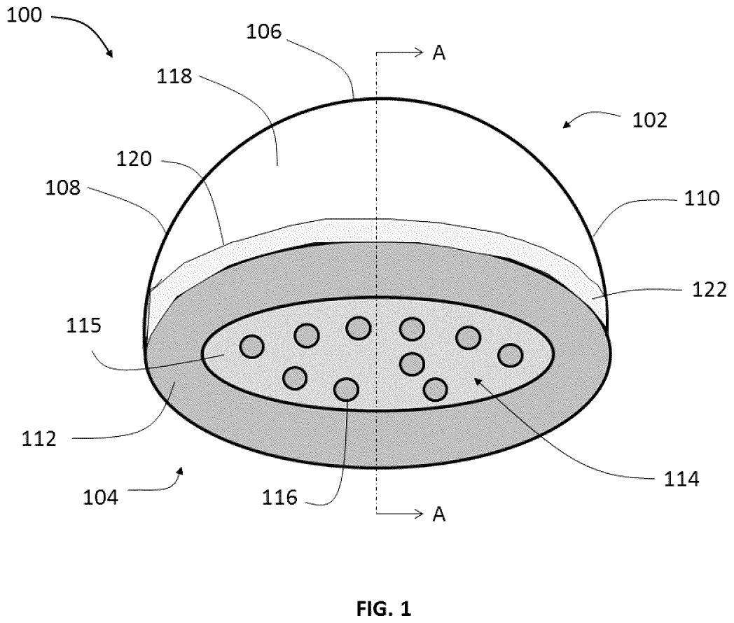

FIG. 1 shows a patellar implant 100 according to an embodiment of the present invention. Patellar implant 100 includes an articulating posterior surface 102 and an opposite anterior surface 104. Posterior surface 102 is symmetrically shaped and includes a median side 106, a lateral side 108 and a medial side 110. The posterior surface is configured to articulate with a distal end portion of a femoral body (not shown). While a symmetrically shaped posterior surface is shown in this embodiment, other embodiments may have anatomically shaped posterior surfaces as described below. Anterior surface 104 is configured to contact a resected patellar bone (not shown). A flat peripheral base 112 is disposed around a conical recess 114. The conical recess tapers and extends towards the posterior surface and includes sidewalls 115. Sidewalls 115 can have projections 116 disposed all across the sidewalls or positioned in strategic locations to improve fixation with resected patellar bone.

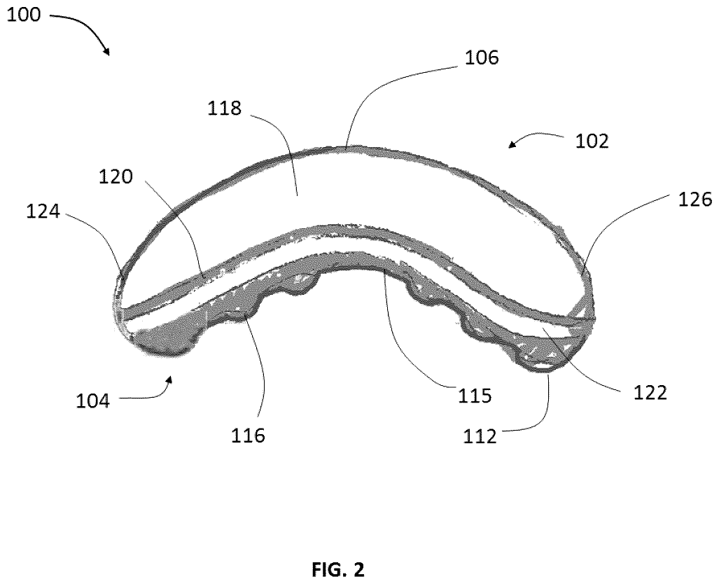

Referring now to FIG. 2, there is shown a cross-section of patellar implant 100. Anterior surface 104 includes a porous exterior layer to allow for attachment and bone ingrowth between the resected patella and patellar implant 100. The porous layer is provided all across the anterior surface including sidewalls 115 of conical recess 114 and projections 116. A solid layer 122 is disposed between anterior surface 104 and posterior surface 106. The solid layer 122 encircles a base of the conical recess 114 as best shown in FIG. 1. The solid layer may be made of a suitable metal, whereas the posterior surface may be made of a polymer. The porous-solid-polymer composition provides patellar implant 100 with ideal strength, bone attachment and femoral kinematic characteristics. Solid layer 122 reinforces the anterior surface of patellar implant 100 and also facilitates attachment with the resected patella by compressing the implant around the natural patellar bone. Polymer surface 118 provides a soft and smooth interface to reduce friction between patellar implant 100 and the femoral body.

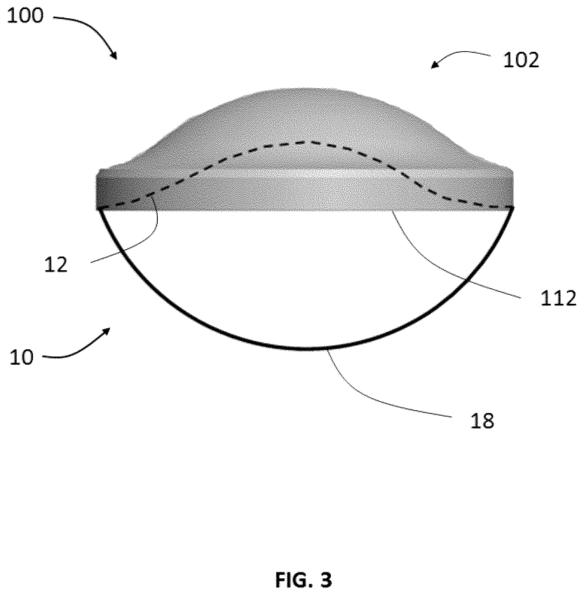

FIG. 3 shows patellar implant 100 positioned on a resected patella 10. Resected patella surface 12 of patella 10 matches anterior surface 104 of patellar implant 100. This allows patellar implant 100 to be seamlessly placed and secured to the resected patella. As shown here, bone removal from patella 10 is minimized, which in turn allows to maximize natural bone retention at the center of the patella. Maximum bone retention at the center of patella 10 allows the natural patellar bone to protect against articulating forces. Minimizing bone removal also ensures that the rigidity of the natural patellar bone construct is maintained after patellar resection. As best seen in FIG. 2, patellar implant 100 has uniform thickness across the implant and thereby distributes forces evenly across the resected patella.

The enlarged anterior surface 104 area, which includes sidewalls 115 of the conical recess 114, provides a much larger contact surface area between patellar implant 100 and patella 10. The sidewalls of conical recess 114 fully encompass and secure at least a portion of resected patella 10 as best shown in FIG. 3. Furthermore, the porous layer, projections 116 and solid layer 122, which is wrapped around the conical recess, further reinforce the attachment between patella 10 and patellar implant 100.

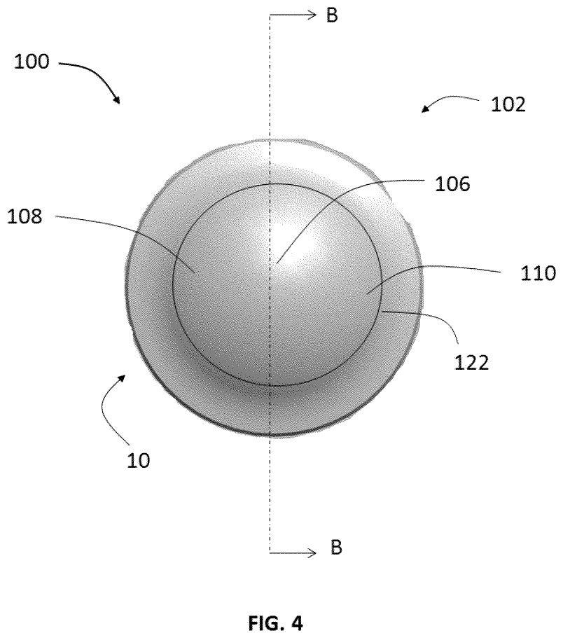

FIG. 4 shows a top view of patellar implant 100 placed over resected patella 10. In this embodiment, patellar implant 10 is sized to completely cover resected patella 10 with no undercap or overhang. Overhang occurs when the footprint of the implant is larger than the footprint of the resected patella, and undercap 136 occurs when the footprint of the implant is smaller than the footprint of the resected patella. Other embodiments may have a patellar implant with an overhang or an undercap depending on implant selection and/or patient specific needs.

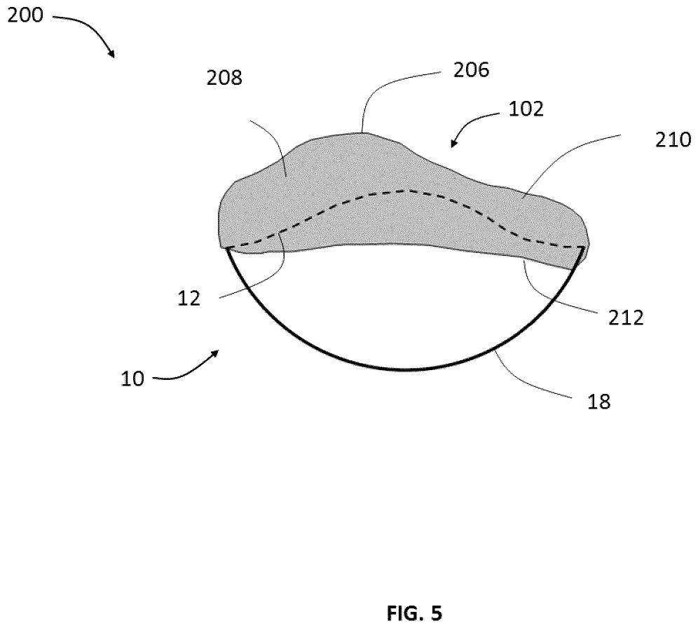

Referring now to FIG. 5, there is shown a patellar implant 200 according to another embodiment of the present invention attached to resected patella 10. Patellar implant 200 is similar to patellar implant 100, and therefore like elements are referred to with similar numerals within the 200-series of numbers. For instance, patellar implant 200 includes flat peripheral base 212 and conical recess 214 with sidewalls 215 (not shown). However, patellar implant has an anatomically-shaped posterior surface 202. Patellar implant 200 has a median ridge 206 separating a medial facet 210 and a lateral facet 208. The anatomically shaped posterior surface allows for improved patellofemoral kinematics. Posterior surface 202 is anatomically shaped to match contours of the articulating surface of a natural patella, and thereby replicates natural patellofemoral kinematics during flexion and extension. Median ridge 206, medial facet 210 and lateral facet 208 may be configured as described in U.S. Provisional Application No. 62/580,575, the entire disclosure of which is incorporated herein by reference.

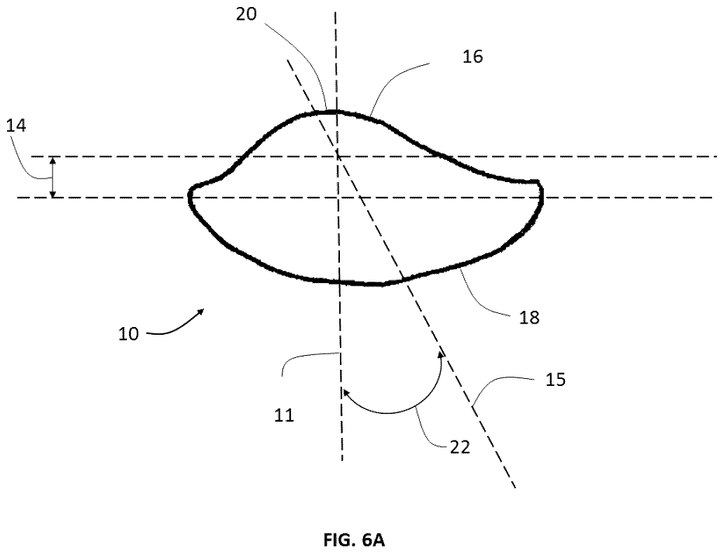

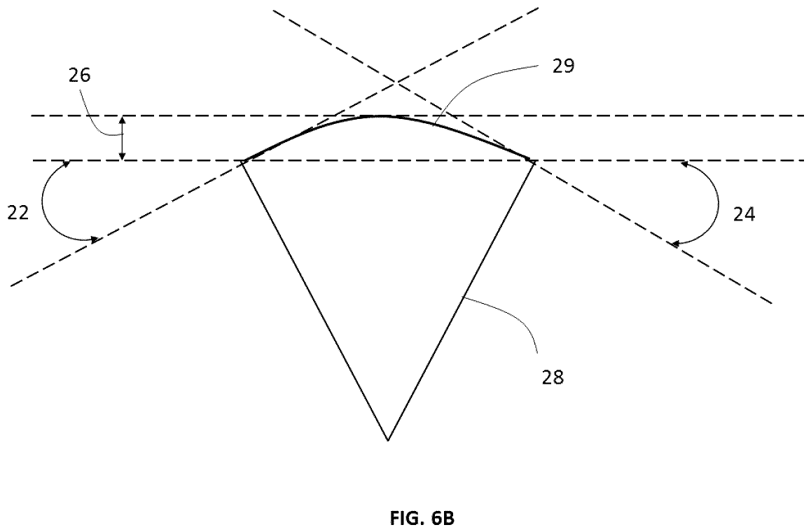

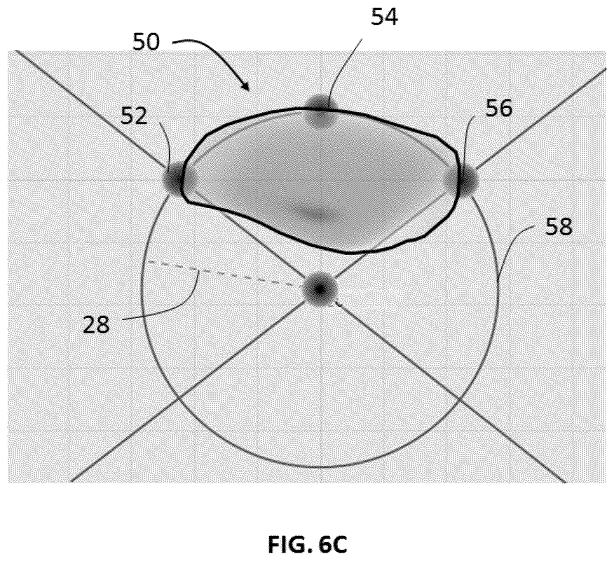

FIGS. 6A and 6B show a method for resecting patella 10 according to another embodiment of the present invention. While the resection method disclosed herein references patellar implant 100, patellar implant 200 or other similar patellar implants may also be used in conjunction with this method. A minimum patella resection depth 14 is first determined as shown in FIG. 6A. Depth 14 denotes the thickness of resected patella 10 which will be encased within the conical recess 114 of patellar implant 100. Minimum depth 14 represents minimum thickness required to prevent detachment of patellar implant 100 from patella 10. Ensuring that only a minimum thickness is resected, i.e., minimum bone removal, allows natural patellar bone retention. A patellar central axis 11 is determined on a patellar model obtained by X-ray, CT scan or other visualization techniques. The patellar model will allow for bony mass determination and the proper location of central axis 11. A patellar tilt axis 15 is determined based on patient specific patellar-femoral geometry and kinematics. A patellar tilt angle 22 defined between central axis 11 and patellar tilt axis 15 denotes patellar depth as shown in FIG. 6A.

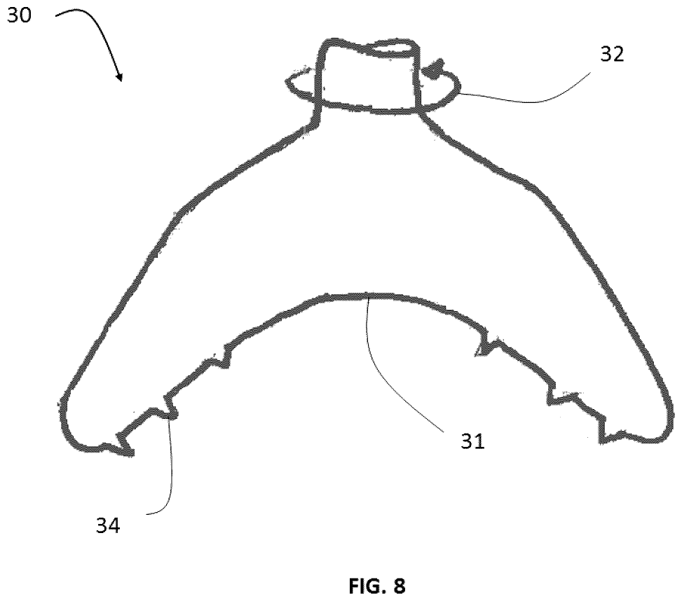

Referring now to FIG. 6B, a patellar surface curvature 29 is determined by medial-lateral slope angles. Optimum medial patellar slope angle 22 and lateral patellar slope angle 24 are determined from the patella model. These values are averaged to compute a radius 28 for a symmetrical conical reamer 30 shown in FIG. 8. Additionally, patient databases for standard patellar sizes and shape may be factored in calculating radius 28 in order to minimize bone removal while simultaneously fitting a predetermined population sample. For example, as shown in FIG. 6C, radius 28 of an unresected patella 50 representing a sample population is determined by locating peripheral edges 52, 54 and 56. A circle 58 joining these peripheral edges is defined by radius 28. Similar measurements across multiple samples can be used to compile a database for standard patellar sizes. By way of example only and not limitation, radius 28 for males is generally around 31.1±3.5mm for females is generally around 27.4±3.3mm based on scans of global population groups.

As shown in FIG. 8, conical reamer 30 includes teeth 34 to cut grooves in patella 10 to maximize surface area for patellar implant 100 fixation. Central curvature 31 of conical reamer 30 is identical to the resected patellar surface curvature 29.

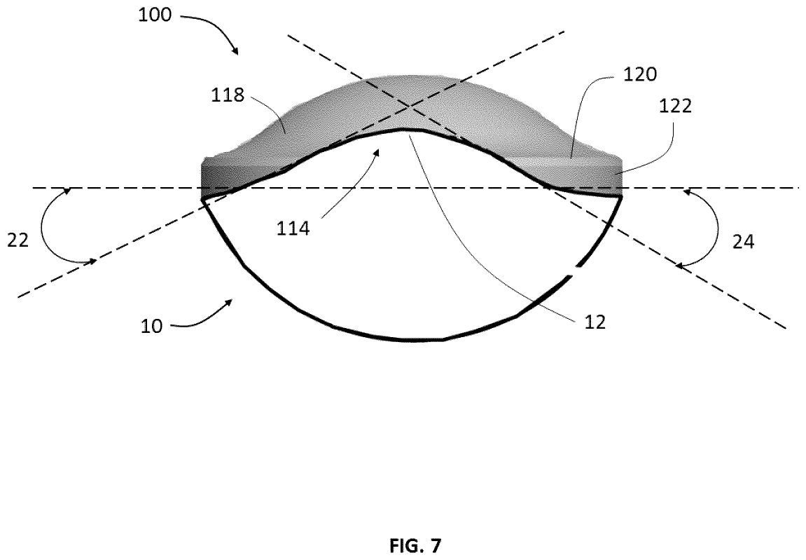

Referring now to FIG. 7, there is shown patellar implant 100 placed on resected patella 10. The conical reamer selection, based on average radius 28, ensures that resected patella surface 12 forms a seamless fit with patellar implant 100. Resected surface 12 corresponds not only to the anterior surface of the recessed patellar implant 100, it also resembles the articulating surface of the natural patella. Hence, bone retention of the natural patella is maximized while ensuring that patellar implant 100 has a generally uniform thickness.

While a patellar implant described in these embodiments have conically shaped anterior surface recesses, other implants may cylindrical, rectangular or similarly shaped recesses. Implants described herein may be made from polymers such as PEEK, carbon fiber reinforced PEEK, PAEK, UHMWPE, metals, ceramics, combinations of the foregoing, or other suitable materials that are biocompatible and possess sufficient strength and rigidity as described above. Additive manufacturing techniques such as 3D printing may be used to fabricate implants of the present invention.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the claims below.

1. A patellar implant comprising:
a posterior articulating surface for engaging a femoral body, and
an anterior surface for engaging a resected patellar bone, the anterior surface opposed to the posterior articulating surface,
wherein the anterior surface includes a recess with a substantially circular opening, the opening defined by the anterior surface disposed around a circumference of the opening, the recess configured to receive and secure the resected patellar bone to the patellar implant.

2. The patellar implant of claim 1, wherein the recess in conically shaped.

3. The patellar implant of claim 1, wherein a thickness of the resected patellar bone is disposed within the recess when the patellar implant is secured to the resected patellar bone, the thickness being defined by a distance between the opening and an apex of the recess.

4. The patellar implant of claim 1, wherein the recess includes at least one projection extending away from the anterior surface.

5. The patellar implant of claim 1, wherein the anterior surface is a porous layer.

6. The patellar implant of claim 5, wherein the posterior articulating surface is a polymeric layer.

7. The patellar implant of claim 6, wherein a solid layer is disposed between the porous layer and the polymeric layer.

8. The patellar implant of claim 1, wherein the posterior articulating surface is symmetrically shaped to the anterior surface.

9. The patellar implant of claim 1, wherein the posterior articulating surface includes a median ridge portion extending posteriorly to and disposed between a lateral portion and a medial portion, wherein the median ridge portion extends along a first length in a superior to inferior direction and along a second length in a medial to lateral direction of the posterior articulating surface, the first length being greater than the second length.

10. The patellar implant of claim 9, wherein the median ridge portion is elliptical in shape.

11. The patellar implant of claim 9, wherein the first length is a major axis and the second length is a minor axis of the median ridge portion respectively.

12. The patellar implant of claim 11, wherein the major axis defines a boundary between a medial side and a lateral side of the median ridge portion, the medial side having one or more curves defined by a curve center located laterally to the major axis, the lateral side having a one or more curves defined by a curve center located medially to the major axis.

13. A patellar implant comprising:
a posterior articulating surface for engaging a femoral body, and
an anterior surface for engaging a resected patellar bone,
wherein the anterior surface includes a recessed portion, a substantially circular opening to the recessed portion defined by the anterior surface, the recessed portion tapering from the opening towards the posterior articulating surface such that the resected patellar bone is received in the recessed portion to secure the patellar implant to the resected patellar bone.

14. The patellar implant of claim 13, wherein the posterior articulating surface includes a median ridge portion extending posteriorly to and disposed between a lateral portion and a medial portion, wherein the median ridge portion extends along a first length in a superior to inferior direction and along a second length in a medial to lateral direction of the posterior articulating surface, the first length being greater than the second length.

15. The patellar implant of claim 14, wherein the median ridge portion is elliptical in shape.

16. The patellar implant of claim 15, wherein the first length is a major axis and the second length is a minor axis of the median ridge portion respectively.

17. The patellar implant of claim 16, wherein the major axis defines a boundary between a medial side and a lateral side of the median ridge portion, the medial side having one or more curves defined by a curve center located laterally to the major axis, the lateral side having a one or more curves defined by a curve center located medially to the major axis.

18. The patellar implant of claim 15, wherein the substantially circular opening to the recessed portion is defined by a flat peripheral base of the anterior surface disposed around a circumference of the opening.

19. The patellar implant of claim 15, wherein the anterior surface is a porous layer.

20. The patellar implant of claim 15, wherein the posterior articulating surface is a polymeric layer.
